(19) United States
(12) Patent Application Publication
(10) Pub. No.: US 2022/0043618 A1
(43) Pub. Date: Feb. 10, 2022

(54) MEDICAL INFORMATION PROCESSING APPARATUS AND MEDICAL INFORMATION PROCESSING METHOD

(71) Applicant: Canon Medical Systems Corporation, Otawara-shi (JP)

(72) Inventors: Taisuke IWAMURA, Utsunomiya (JP); Koichi TERAI, Shioya (JP); Kazutoshi YANAGIDA, Otawara (JP)

(73) Assignee: Canon Medical Systems Corporation, Otawara-shi (JP)

(21) Appl. No.: 17/389,920

(22) Filed: Jul. 30, 2021

(30) Foreign Application Priority Data
Aug. 6, 2020 (JP) 2020-133877

(51) Int. Cl. G06F 3/14; G06T 11/00; H04L 29/08; G16H 30/20; G16H 30/40; G16H 40/20

(52) U.S. Cl. CPC G06F 3/14; G06T 11/001; H04L 67/12; G06T 2210/41; G16H 30/40; G16H 40/20; G16H 30/20

(57) ABSTRACT

According to one embodiment, a medical information processing apparatus includes processing circuitry. The processing circuitry generates progress information on a communication process, based on an anatomical site of a subject included in a communication-target medical image, in the communication process of the medical image related to the subject. The processing circuitry displays the progress information. The processing circuitry terminates the communication process in response to a trigger that is a signal for terminating the communication process.

### CROSS-REFERENCE TO RELATED APPLICATIONS

This application is based upon and claims the benefit of priority from Japanese Patent Application No. 2020-133877, filed Aug. 6, 2020, the entire contents of which are incorporated herein by reference.

### FIELD

Embodiments described herein relate generally to a medical information processing apparatus and a medical information processing method.

### BACKGROUND

In the recent medical field, a product (analysis server) that analyzes a received medical image and detects observations such as a lesion included in the medical image is being developed.

In general, an analysis server acquires medical images from a medical image diagnostic apparatus or PACS (Picture Archiving and Communication Systems) server by communications based on the DICOM (Digital Imaging and Communications in Medicine) standard, and performs analysis. Specifically, the analysis server establishes an association with reference to a medical image storage source in order to perform the DICOM communications. Next, the analysis server acquires medical images to be analyzed from the storage source. Subsequently, the analysis server releases the association and analyzes the acquired medical images. In this manner, the analysis server handles the medical images acquired in one association as a unit and analyzes them at the end of the association.

However, where the analysis server acquires a large number of medical images in one association, the analysis cannot be started until all medical images have been received. For example, in order to analyze the condition of a patient with suspected brain damage, the analysis server has to receive the patient's whole-body CT (Computed Tomography) images in one association, and the analysis cannot be started until all CT images have been received. As a result, the analysis of urgent brain damage such as an acute cerebral infarction may be delayed and the prognosis of the patient may worsen.

### BRIEF DESCRIPTION OF THE DRAWINGS

FIG. 1 is a diagram illustrating a configuration of a medical information processing apparatus according to a first embodiment.

FIG. 2 is a diagram illustrating how a medical information processing system operates according to the first embodiment.

FIG. 3 is a diagram illustrating how the medical information processing apparatus operates according to the first embodiment.

FIGS. 4A and 4B are diagrams illustrating a first example of how progress information is displayed.

FIGS. 5A and 5B are diagrams illustrating a second example of how the progress information is displayed.

FIG. 6 is a diagram illustrating how a medical information processing system operates according to a second embodiment.

FIG. 7 is a diagram illustrating how a medical information processing apparatus operates according to the second embodiment.

FIG. 8 is a diagram illustrating how a medical information processing system operates according to a third embodiment.

FIG. 9 is a diagram illustrating how a medical information processing apparatus operates according to the third embodiment.

### DETAILED DESCRIPTION

In general, according to one embodiment, a medical information processing apparatus includes processing circuitry. The processing circuitry generates progress information on a communication process, based on an anatomical site of a subject included in a communication-target medical image, in the communication process of the medical image related to the subject. The processing circuitry displays the progress information. The processing circuitry terminates the communication process in response to a trigger that is a signal for terminating the communication process.

Hereinafter, a medical information processing apparatus and a medical information processing method according to the embodiment will be described with reference to the accompanying drawings. In the embodiments described below, elements assigned with the same reference symbols perform the same operation, and a redundant description of such elements will be omitted as appropriate.

### First Embodiment

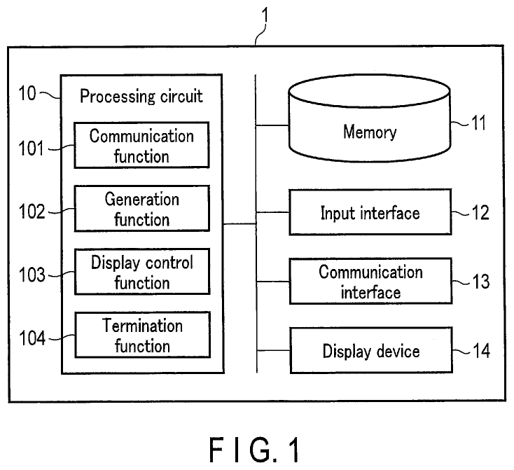

A configuration of a medical information processing apparatus 1 according to the first embodiment will be described with reference to FIG. 1. FIG. 1 is a block diagram illustrating the configuration of the medical information processing apparatus 1 according to a first embodiment.

The medical information processing apparatus 1 includes a processing circuit 10, a memory 11, an input interface 12, a communication interface 13 and a display device 14. These components are communicably coupled to each other via a bus. It should be noted that each component does not have to be individual hardware. For example, at least two of the components may be integrated as one hardware element.

The processing circuit 10 controls the operation of the medical information processing apparatus 1. The processing circuit 10 includes hardware processors such as a CPU (Central Processing Unit), an MPU (Micro Processing Unit), a GPU (Graphics Processing Unit) and an FPU (Floating Point Unit). By executing each of programs expanded in the memory 11 by means of the processor, the processing circuit 10 executes each of the functions (namely, a communication function 101, a generation function 102, a display control function 103 and a termination function 104) corresponding to the programs. It should be noted that each function does not have to be realized by a processing circuit consisting of a single processor. For example, each function may be realized by a processing circuit that combines a plurality of processors.

The communication function 101 performs a communication process of medical images related to a subject. The communication process includes a transmission process and a reception process related to medical images. In addition, the communication function 101 establishes and releases an association with reference to an external device.

In the communication process of medical images related to the subject, the generation function 102 generates progress information on the communication process, based on what anatomical site of the subject is included in communication-target medical images.

The display control function 103 displays the progress information.

The termination function 104 terminates the communication process in response to a trigger. The trigger is a signal for terminating the communication process, and includes a case where an input is entered by a user or a case where a predetermined condition is satisfied. For example, the condition is that the communication-target medical images contain a site other than a specific anatomical site of the subject.

The memory 11 stores information, such as data, programs and algorithms that are used by the processing circuit 10. The memory 11 includes a semiconductor memory element such as a RAM (Random Access Memory), as hardware. The memory 11 may be a drive device that reads and writes information to and from an external storage device, such as a magnetic disk (a floppy (registered trademark) disk or a hard disk), a magneto-optical disk (MO), an optical disk (CD, DVD or Blu-ray (registered trademark)), a flash memory (a USB flash memory, a memory card, or SSD) or a magnetic tape. The storage area of the memory 11 may be inside the medical information processing apparatus 1 or may be inside an external storage device.

The input interface 12 receives an input from an operator (a user), converts the received input into an electric signal, and outputs the received input to the processing circuit 10. As the input interface 12, a physical operation device, such as a mouse, a keyboard, a trackball, a switch, a button, a joystick, a touch pad or a touch panel display, can be used. The input interface 12 may be replaced with a device that receives an input from an external input device separate from the medical information processing apparatus 1, converts the received input into an electric signal, and outputs the electric signal to the processing circuit 10.

The communication interface 13 transmits and receives data to and from an external apparatus. Any communication standard can be used for the communications between the communication interface 13 and the external device. For example, HL7 (Health Level 7) can be used for communications related to medical textual information, and DICOM can be used for communications related to medical image information.

The display device 14 displays information, such as data generated by the processing circuit 10 and data stored in the memory 11. As the display device 14, for example, a display such as a cathode ray tube (CRT) display, a liquid crystal display (LCD), a plasma display, an organic electro-luminescence display (OELD), a tablet terminal, etc. can be used.

The medical information processing apparatus 1 is used in a system (medical information processing system) for data communications of medical images etc. between a sender such as a medical image diagnostic device or a PACS server and a receiver such as an analysis server. There are a case where the medical information processing apparatus 1 functions independently of other apparatuses (first embodiment), a case where it functions while being incorporated in the sender of data (second embodiment) and a case where it functions while being incorporated in the receiver of data (third embodiment). A description will be given of how the medical information processing apparatus 1 operates according to each embodiment.

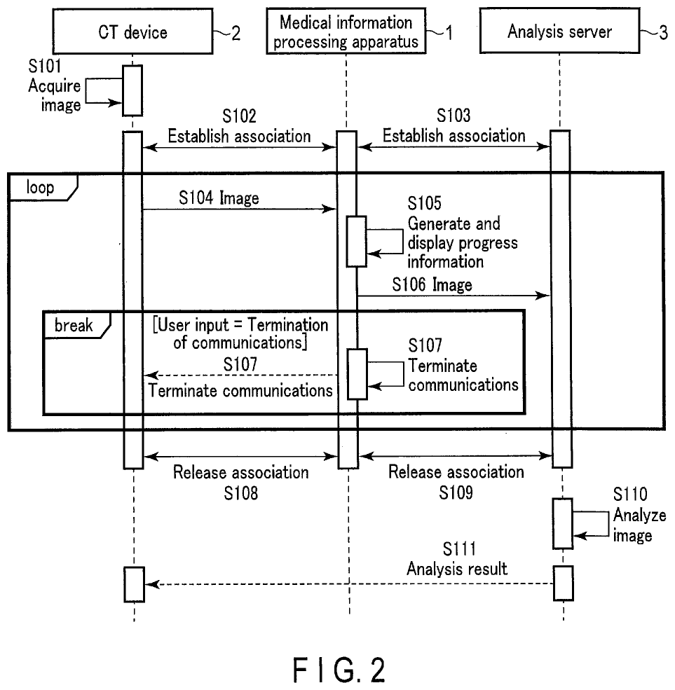

An operation of the medical information processing system according to the first embodiment will be described with reference to FIG. 2. FIG. 2 is a sequence diagram illustrating how the medical information processing system operates according to the first embodiment. In the medical information processing system according to the first embodiment, the medical information processing apparatus 1 exists independently of a CT device 2 and an analysis server 3, the former of which is an example of the medical image diagnostic apparatus.

In step S101, the CT device 2 acquires a medical image of a subject. For example, the CT device 2 acquires a whole-body CT image of the subject by taking a whole-body CT image of the subject. The whole-body CT image includes a large number of two-dimensional cross-sectional images (axial cross-sections) from the head to the foot of the subject. The image type is not limited to the CT image, and may be an image type such as an MR (Magnetic Resonance) image, a nuclear medicine image, an ultrasonic image, an endoscopic image, or a simple X-ray image. Further, the CT device 2 may acquire a large number of medical images stored in its own memory instead of taking images of the subject. That is, a large number of medical images related to the subject may be acquired as any image type and by any means.

In step S102, the CT device 2 establishes an association for DICOM communications with reference to the medical information processing apparatus 1. For example, the CT device 2 requests the medical information processing apparatus 1 to establish an association, and the medical information processing apparatus 1 makes an association establishment response. At this time, the CT device 2 and the medical information processing apparatus 1 confirm each other's IP (Internet Protocol) address, port number, AE (Application Entity) title, SOP (Service Object Pair) class etc., so as to establish the association.

In step S103, the medical information processing apparatus 1 establishes an association for DICOM communications with reference to the analysis server 3. For example, the medical information processing apparatus 1 requests the analysis server 3 to establish an association, and the analysis server 3 makes an association establishment response. At this time, the medical information processing apparatus 1 and the analysis server 3 confirm each other's IP address, port number, AE title, SOP class, etc., so as to establish the association. It should be noted that steps S102 and S103 may be executed at the same time, or either of the steps may be executed first. As a result, the CT device 2 can transmit the acquired medical image to the analysis server 3 via the medical information processing apparatus 1.

In step S104, the CT device 2 transmits the medical image to the medical information processing apparatus 1. For example, the CT device 2 transmits one of the untransmitted whole-body CT images that shows the uppermost portion of the head (top of the head). It should be noted that step S104 is repeated until all medical images are transmitted, unless the CT device 2 receives a communication termination instruction. That is, in the present example, images are transmitted one by one from the image showing the top of the head to the image showing the lowermost end of a foot (sole).

At this time, the medical images transmitted from the CT device 2 to the analysis server 3 via the medical information processing apparatus 1 are defined as communication-target medical images. In other words, the communication-target medical images are medical images transmitted and received between different apparatuses. After the end of the communications, the communication-target medical images are referred to as medical images that have been communicated. Thereafter, when the communications of next medical images are started, these are referred to as the next communication-target medical images. That is, in one association, a medical image is communicated a plurality of times, and each time the communications of the medical images are completed in one association, the next medical images are referred to as the next communication-target medical images.

In step S105, the medical information processing apparatus 1 generates and displays progress information on the communication process, based on an anatomical site of the subject included in received medical images (communication-target medical images). For example, the medical information processing apparatus 1 detects an anatomical site of the subject by applying a detection algorithm to the received medical images. Subsequently, based on the detected anatomical site, which is a detection result, progress information on the communication process at the current time is generated and displayed. Each time step S105 is repeatedly executed, newly generated progress information may be updated by overwriting already generated progress information.

In step S106, the medical information processing apparatus 1 transmits received medical images (communication-target medical images) to the analysis server 3. For example, the medical information processing apparatus 1 transmits the received CT images to the analysis server 3. By completing the series of processes in steps S104 to s106 in this manner, the communications of the medical images of one communication target are completed.

In step S107, the medical information processing apparatus 1 determines whether or not a communication termination instruction has been received from the user. The communication termination instruction is input to the medical information processing apparatus 1 by the user at an arbitrary time while the medical information processing apparatus 1 repeatedly executes steps S104 to S106. The input instruction waits for determination until it is processed in step S107. When the medical information processing apparatus 1 determines that the communication termination instruction has been received, the communications of the medical images are terminated. Specifically, the medical information processing apparatus 1 transmits to the CT device 2 the communication termination instruction for releasing the association established in step S102. Simultaneously, the medical information processing apparatus 1 executes the communication termination process for releasing the association established in step S103. The medical information processing apparatus 1 may terminate the communication of medical images at the time when the communication termination instruction is input. On the other hand, where the medical information processing apparatus 1 determines that a communication termination instruction is not received, step S107 is not executed and the process returns to step S104.

In step S108, the CT device 2 releases the association with reference to the medical information processing apparatus 1, based on the received communication termination instruction. For example, the CT device 2 requests the medical information processing apparatus 1 to release the association, and the medical information processing apparatus 1 makes an association release response. The CT device 2 may release the association without receiving the response from the medical information processing apparatus 1.

In step S109, the medical information processing apparatus 1 releases the association with reference to the analysis server 3 by executing the communication termination process. For example, the medical information processing apparatus 1 requests the analysis server 3 to release the association, and the analysis server 3 makes an association release response. The medical information processing apparatus 1 may release the association without receiving the response from the analysis server 3. It should be noted that steps S108 and S109 may be executed at the same time, or either of the steps may be executed first. Thus, the analysis server 3 can perform image analysis on medical images acquired in units of one association starting from the establishment of the association to the release thereof.

In step S110, the analysis server 3 performs image analysis on the acquired medical images. For example, the analysis server 3 analyzes the acquired CT images by applying an analysis algorithm for detecting observations of an acute cerebral infarction. At this time, the analysis may be performed on each of the acquired CT images, or on volume data generated from all or some of the acquired CT images. That is, the image analysis may be performed in any desirable manner.

In step S111, the analysis server 3 sends an analysis result to the CT device 2. For example, the analysis server 3 sends to the CT device 2 a composite image in which a region suspected of having an acute cerebral infarction is superimposed on an acquired CT image as an analysis result. Where the analysis result has to be sent by DICOM communications, the analysis server 3 may establish an association with reference to the CT device 2. Further, the analysis server 3 may send the analysis result to the CT device 2 via the medical information processing apparatus 1.

The operation of the medical information processing system according to the first embodiment has been described above. Of all processes (steps S101 to S111) performed by the medical information processing system of the first embodiment, steps S102 to S109 are executed by the medical information processing apparatus 1. A detailed description to will be given of the processes executed by the medical information processing apparatus 1.

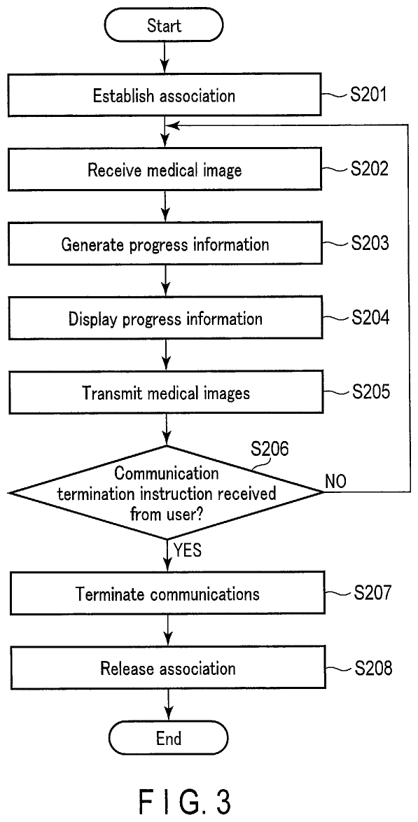

An operation of the medical information processing apparatus according to the first embodiment will be described with reference to FIG. 3. FIG. 3 is a flowchart illustrating how the medical information processing apparatus operates according to the first embodiment.

In step S201, the medical information processing apparatus 1 executes the communication function 101 to establish an association for DICOM communications between the CT device 2 and the analysis server 3 (corresponding to steps S102 and S103). Specifically, the IP address, port number, AE title, SOP class, etc. are confirmed with respect to the devices between which communications are to be performed via the communication interface 13, and the association is established.

In step S202, the medical information processing apparatus 1 executes the communication function 101 to receive a medical image (a communication-target medical image) transmitted from the CT device 2. Specifically, the medical image transmitted by the CT device 2 is received via the communication interface 13 and stored in the memory 11. It should be noted that step S202 is a step executed between steps S104 and S105 but is not illustrated in FIG. 2 for the sake of simplicity.

In step S203, the medical information processing apparatus 1 executes the generation function 102 to generate progress information on the communication process, based on an anatomical site included in received medical images (communication-target medical images) (this process corresponds to step S105). For example, an algorithm for detecting a specific anatomical site (for example, the head) is applied to medical images stored in the memory 11 so as to detect the specific anatomical site. A detection result may be region information regarding the detected anatomical site or may be textual information. Thereafter, progress information on the communication process at the current time is generated based on the detection result. The generated progress information is associated with medical images for which the progress information is generated and stored in the memory 11. That is, the progress information is associated with the received medical images (communication-target medical images).

The applied algorithm may detect brain regions included in the head, such as the cerebrum, the diencephalon, the cerebellum and the brain stem. The applied algorithm may detect not only the head but also body surface regions of the neck, trunk, limbs, etc. That is, an algorithm for detecting an arbitrary anatomical site at an arbitrary anatomical classification level may be applied.

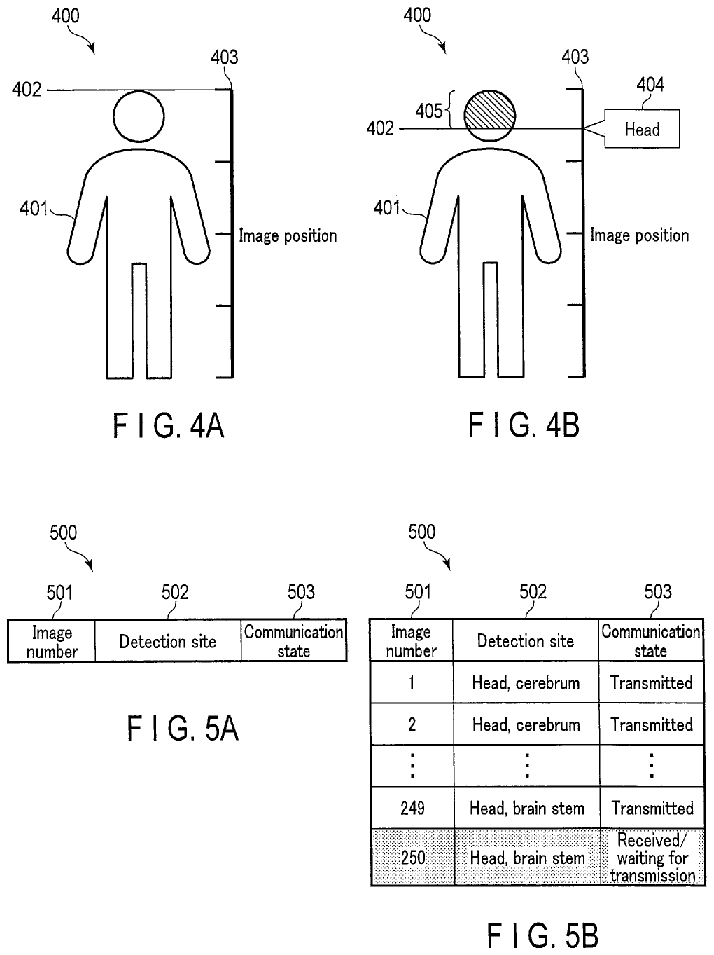

In step S204, the medical information processing apparatus 1 displays progress information by executing the display control function 103 (the process corresponds to step S105). Specifically, the progress information associated with the medical images stored in the memory 11 is displayed on the display device 14. An example of how the progress information is displayed will be described later with reference to FIGS. 4 and 5.

In step S205, the medical information processing apparatus 1 executes the communication function 101 to transmit medical images (communication-target medical images) received from the CT device 2 to the analysis server 3 (the process corresponds to step S106). Specifically, the medical images stored in the memory 11 are transmitted via the communication interface 13. By completing the series of processes in steps S202 to S205 in this manner, the communications of the medical images of one communication target are completed.

In step S206, the medical information processing apparatus 1 executes the termination function 104 to determine whether or not a communication termination instruction has been received from the user (the process corresponds to step S107). The communication termination instruction is input to the medical information processing apparatus 1 via the input interface 12 by the user at an arbitrary time while the medical information processing apparatus 1 repeatedly executes steps S202 to S205. The input instruction waits for determination until it is processed in step S206. Where the medical information processing apparatus 1 determines that the communication termination instruction has been received (YES in step S206), the process proceeds to step S207. Where it is determined that the instruction has not been received (NO in step S206), the process returns to step S202.

In step S207, the medical information processing apparatus 1 executes the termination function 104 to terminate the communications of medical images, with the reception of the communication termination instruction from the user as a trigger (the process corresponds to step S107). If the communication termination instruction is received during the processes of steps S202 to S205, the medical information processing apparatus 1 may interrupt the processes and terminate the communications of medical images at the time when the communication termination instruction is input.

In step S208, the medical information processing apparatus 1 executes the communication function 101 to release the association between the CT device 2 and the analysis server 3 (the process corresponds to steps S108 and S109). Specifically, the association with reference to the device for which communications are performed via the communication interface 13 is released.

The operation of the medical information processing apparatus according to the first embodiment has been described above. A description will be given of an example of how progress information is displayed on the display device 14 of the medical information processing apparatus 1.

A first example of how the progress information is displayed will be described with reference to FIG. 4. FIG. 4 is a schematic diagram illustrating a first example of how the progress information is displayed. FIG. 4 is divided into FIGS. 4A and 4B in order to illustrate an example of how the progress information is displayed changes with time. FIG. 4 and FIG. 5 illustrate how the progress information is displayed, referring to a whole-body CT image including a large number of two-dimensional cross-sectional images (axial cross-sectional images) from the head to the foot of the subject.

FIG. 4A is a diagram illustrating how an anatomical atlas 400 is before the medical information processing apparatus 1 displays first progress information (initial state). What is shown in FIG. 4A may be displayed on the display device 14 in advance before the progress information on a first received medical image is displayed (before step S204).

The anatomical atlas 400 is a diagram in which an anatomical diagram relating to general anatomical sites of a human body and various additional information are defined in the same three-dimensional space. The anatomical atlas 400 includes a human body model 401, a bar 402 and a scale 403.

The human body model 401 is a three-dimensional model in which general anatomical sites of the human body are defined. For example, the human body model 401 may be an anatomical model in which body sites such as muscles, a skeleton, nerves and organs are defined. The human body model 401 is not limited to this but may be an anatomical model in which image types, such as CT images, MR images, nuclear medicine images, ultrasonic images, endoscopic images and simple X-ray images, are defined in arbitrary cross sections. Further, the human body model 401 may be a two-dimensional model. Any model can be used as the human body model 401.

The bar 402 is a straight line representing where in the anatomical atlas 400 a communication-target medical image is taken. In FIG. 4A, the first progress information has not yet been displayed, so that the bar 402 does not have to be displayed; alternatively, it may be displayed along a horizontal plane in contact with the top of the head of the human body model 401 as an initial position. One end of the bar 402 may intersect the scale 403.

The scale 403 is a graduated straight line for associating the position of a communication-target medical image with the human body model 401. In conjunction with the bar 402, the scale 403 represents a position where in the human body model 401 a communication-target medical image is taken. For example, the scale 403 is displayed by the length from the head top to the soles of feet along the body axis direction of the human body model 401. The scale 403 may be displayed at a position where it does not overlap the human body model 401.

FIG. 4B is a diagram illustrating how the anatomical atlas 400 is when the medical information processing apparatus 1 displays progress information. The progress information is generated based on the anatomical site of the subject included in the communication-target medical image, and is displayed with additional information on the human body model 401, such as a balloon 404 and a communicated region 405 as well as the bar 402 and the scale 403.

The bar 402 represents a position where in the anatomical atlas 400 the communication-target medical image is being taken currently. In FIG. 4B, the bar 402 is displayed in a horizontal plane that passes through the head of the human body model 401. The bar 402 moves from above to below of the scale 403 as the anatomical site of the subject detected from the communication-target medical image changes from the head to the foot. In this manner, the bar 402 graphically displays the position of the communication-target medical image in real time.

The textual information “head” is indicated in the balloon 404 as the anatomical site (detection result) detected from the communication-target medical image. The balloon 404 is displayed in the vicinity of the location where the bar 402 intersects the scale 403 and moves in relation to the location. The balloon 404 indicates the anatomical site of the communication-target medical image by textual information and moves as the bar 402 moves along the scale 403.

The region of the human body model 401 (communicated region 405) corresponding to a medical image (a communicated medical image) for which communications have been completed up to the current time is colored in a color different from that of the remaining regions of the human body model 401. As the communicated region 405, the region (head) of the human body model 401 located above the bar 402 is colored in the different color. As the bar 402 moves from above to below of the human body model 401, the communicated region 405 also gradually expands from above to below. In this manner, the communicated region 405 graphically displays the position of the communicated medical image in real time.

It should be noted that the scale 403 does not have to be displayed, and only the bar 402 and human body model 401 may be displayed. Alternatively, the human body model 401 does not have to be displayed, and one-dimensional information indicating the position of a communication-target medical image may be displayed on the scale 403, as in a progress bar relating to the Internet communications.

In order to display a communication-target medical image in association with a specific position of the human body model 401, based on the region information on a detected specific anatomical site, cross-sectional images of various anatomical sites are defined for the respective cross sections of the human body model 401. For example, where a brainstem region is detected in a communication-target medical image, a cross section of the human body model 401 that includes a brainstem region morphologically similar to the detected brainstem region is specified. The position of the specified cross section of the human body model 401 may be regarded as a position corresponding to the communication-target medical image.

The first example of how the progress information is displayed has been described so far. As described above, in the progress information on the example illustrated in FIG. 4, the progress information on the communication process is displayed on the anatomical atlas 400 by means of the bar 402, the scale 403, the balloon 404 and the communicated region 405. By looking at the bar 402 and the balloon 404, the user can determine to which anatomical site of the subject the communication-target medical image currently corresponds. Further, by looking at the communicated region 405, the user can determine to which medical image the communications have been completed.

A second example of how the progress information is displayed will be described with reference to FIG. 5. FIG. 5 is a table illustrating the second example of how the progress information is displayed. FIG. 5 is divided into FIGS. 5A and 5B in order to illustrate an example of how the progress information is displayed changes with time. FIGS. 5A and 5B are examples of how the progress information is at the same points of time as FIGS. 4A and 4B, respectively.

FIG. 5A is a diagram illustrating how a progress information table 500 is before the medical information processing apparatus 1 displays first progress information (initial state). What is shown in FIG. 5A may be displayed on the display device 14 in advance before the progress information on a first received medical image is displayed (before step S204).

The progress information table 500 has three headers (an image number 501, a detection site 502 and a communication state 503). In the progress information table 500, a plurality of records each including a set consisting of the image number 501, the detection site 502 and the communication state 503 are stored. One record is created each time the communications of the medical images of one communication target are completed. In FIG. 5A, a state before first progress information is displayed is illustrated, and no record is created.

The image number 501 is an identifier assigned to a communication-target medical image. For example, the medical information processing apparatus 1 assigns an image number 501 to each of the medical images received in step S202. Specifically, as the image number 501, “1” is assigned to a first received medical image, and the numbers incremented by 1 such as “2”, “3”, etc. are assigned to the medical images received thereafter.

The detection site 502 is information representing a detection result obtained by applying a detection algorithm to a communication-target medical image. The detection site 502 is the result of detecting a specific anatomical site in a communication-target medical image in step S203, and is stored in association with the image number 501 of the medical image for which the detection is performed.

The communication state 503 is information representing the communication state of the medical image. The communication state 503 is displayed, for example, by two types of items: “received/waiting for transmission” which indicates that a medical image has been received but not yet been transmitted, and “transmitted” which indicates that a medical image has already been transmitted.

FIG. 5B is a diagram illustrating how the progress information table 500 is when the medical information processing apparatus 1 displays progress information. Specifically, FIG. 5B illustrates a state where the detection and transmission have been completed up to the medical image whose image number 501 is “249”, and where the detection has been completed and the transmission has not yet been performed only for the medical image whose image number 501 is “250”.

With respect to the medical images for which the detection and transmission have been completed, the name of the anatomical site detected as the detection site 502 is displayed, and “transmitted” is displayed as the communication state 503. For example, “head, cerebrum” is stored as the detection site 502 related to the medical image whose image number 501 is “1”, and “transmitted” is stored as the communication state 503 in association with the detection site 502. On the other hand, with respect to the medical image for which the detection has been completed but which has not yet been transmitted, the name of the anatomical site detected as the detection site 502 is displayed, and “received/waiting for transmission” is displayed as the communication state 503. For example, “head, brain stem” is stored as the detection site 502 of the medical image whose image number 501 is “250”, and “received/waiting for transmission” is stored as the communication state 503 in association with the detection site 502. If a detection result is not known, “not detected” may be displayed as the detection site 502.

In FIG. 5B, the medical image whose image number 501 is “250” corresponds to a communication-target medical image, and the medical images whose image numbers 501 are “1” to “249” correspond to already communicated medical images. The record related to the communication-target medical image may be highlighted in a color different from that of the other records.

The second example of how the progress information is displayed has been described so far. As described above, in the display example of progress information illustrated in FIG. 5, the progress information on the communication process is displayed as the progress information table 500. By looking at the detection site 502 included in the record of the communication-target medical image, the user can determine to which anatomical site of the subject the communication-target medical image currently corresponds. Further, by looking at the records other than the communication-target medical image, the user can determine to which medical image the communications have been completed. Further, since the record related to the communication-target medical image is highlighted, the user can easily identify the record related to the communication-target medical image from among all records.

A plurality of examples of how the progress information is displayed have been described above. The user inputs a communication termination instruction to the medical information processing apparatus 1 at a desired point of time while referring to the displayed progress information. For example, when it is desired to cause the analysis server 3 to immediately start image analysis regarding the observations of the head, a communication termination instruction is input when the progress information on the communication-target medical image has been switched from the head to the neck. Thus, the communication and association of the medical image terminate, so that the analysis server 3 can immediately start the image analysis on the specific anatomical site at an arbitrary point of time in response to the user input.

The medical information processing apparatus 1 may issue a communication termination instruction not by using the user input as a trigger but by using a predetermined condition as a trigger. The condition may be, for example, a case where a communication-target medical image includes a site other than a specific anatomical site of the subject. For example, where the condition is set such that “communication terminates when the communication-target medical image is an image other than that of the head”, the medical information processing apparatus 1 automatically makes determination and issues a communication termination instruction when the communication-target medical image is switched from the head to the neck. In this manner, the user does not have to manually input the communication termination instruction.

### Second Embodiment

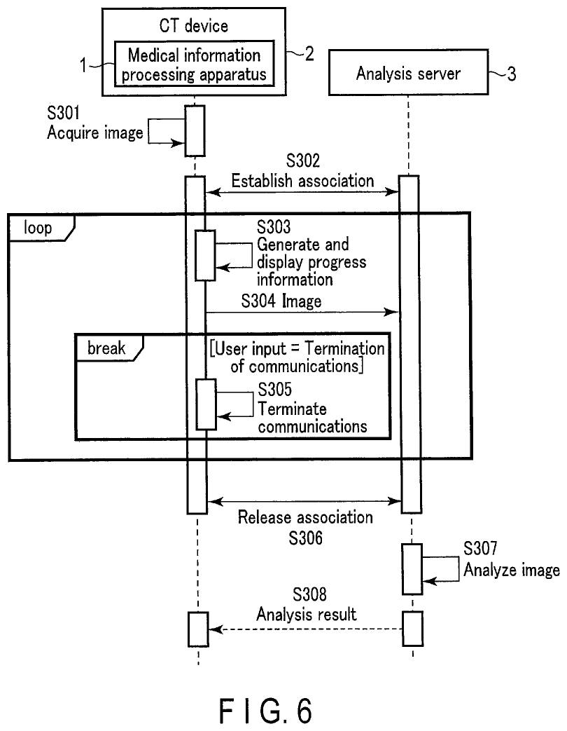

An operation of the medical information processing system according to the second embodiment will be described with reference to FIG. 6. FIG. 6 is a sequence diagram illustrating how the medical information processing system operates according to the second embodiment. In the second embodiment, the medical information processing apparatus 1 is incorporated in the CT device 2, which is the sender of medical images. At this time, the medical information processing apparatus 1 displays progress information on the medical image transmission process of the CT device 2, and terminates the transmission process of the CT device 2 in response to a trigger. The CT device 2 may independently execute all operations of the medical information processing apparatus 1.

In step S301, the CT device 2 acquires a medical image of a subject. Step S301 is similar to step S101.

In step S302, the CT device 2 establishes an association for DICOM communications with reference to the analysis server 3. For example, the CT device 2 requests the analysis server 3 to establish the association, and the analysis server 3 makes an association establishment response. At this time, the CT device 2 and the analysis server 3 confirm each other's IP address, port number, AE title, SOP class, etc., so as to establish the association. As a result, the CT device 2 can transmit the acquired medical image to the analysis server 3.

In step S303, the medical information processing apparatus 1 generates and displays progress information on the transmission process, based on the anatomical site of the subject included in an untransmitted medical image included among the medical images the CT device 2 acquires in step S301. For example, the medical information processing apparatus 1 detects an anatomical site of the subject by applying a detection algorithm to one untransmitted medical image the CT device 2 acquires. Subsequently, based on the detected anatomical site, which is a detection result, progress information on the transmission process is generated and displayed. Each time step S303 is repeatedly executed, newly generated progress information may be updated by overwriting already generated progress information.

In step S304, the CT device 2 transmits the medical image to the analysis server 3. For example, the medical information processing apparatus 1 transmits one untransmitted medical image, which is a transmission-target medical image, to the analysis server 3. By completing the series of processes in steps S303 to S304 in this manner, the transmission of the one untransmitted medical image is completed. The processes of step S303 and step S304 may be interchanged. That is, one untransmitted medical image may first be transmitted to the analysis server 3, and then progress information on the transmission process may be generated and displayed based on the anatomical site of the untransmitted medical image.

In step S305, the medical information processing apparatus 1 determines whether or not a communication termination instruction has been received from the user. The communication termination instruction is input to the medical information processing apparatus 1 by the user at an arbitrary time while the medical information processing apparatus 1 repeatedly executes steps S303 to S304. The input instruction waits for determination until it is processed in step S305. Where the medical information processing apparatus 1 determines that the communication termination instruction has been received, the transmission of medical images by the CT device 2 is terminated. Specifically, the medical information processing apparatus 1 executes a transmission termination process for releasing the association the CT device 2 establishes in step S302. The medical information processing apparatus 1 may terminate the transmission of the medical images at the time when the communication termination instruction is input. On the other hand, where it is determined that the instruction is not received, the medical information processing apparatus 1 does not execute step S305, and the process returns to step S303.

In step S306, the CT device 2 releases the association with reference to the analysis server 3 by executing the transmission termination process. For example, the CT device 2 requests the analysis server 3 to release the association, and the analysis server 3 makes an association release response. The CT device 2 may release the association without accepting the response of the analysis server 3. Thus, the analysis server 3 can perform image analysis on medical images acquired in units of one association starting from the establishment of the association to the release thereof.

In step S307, the analysis server 3 performs image analysis on the acquired medical images. Step S307 is similar to step S110.

In step S308, the analysis server 3 sends an analysis result to the CT device 2. Step S308 is similar to step S111.

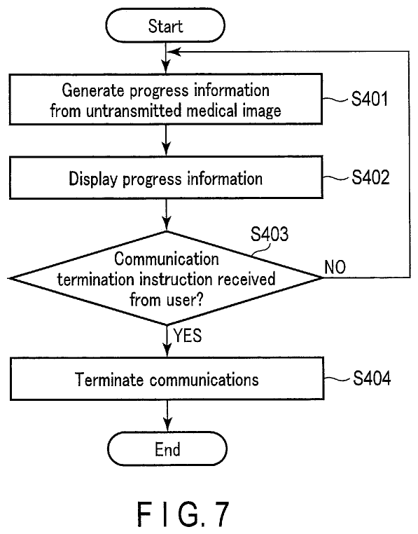

An operation of the medical information processing apparatus according to the second embodiment will be described with reference to FIG. 7. FIG. 7 is a flowchart illustrating how the medical information processing apparatus operates according to the second embodiment.

In step S401, the medical information processing apparatus 1 executes the generation function 102 to generate progress information on the transmission process, based on an anatomical site included in an untransmitted medical image, which is a transmission-target medical image (this process corresponds to step S303). For example, an algorithm for detecting a specific anatomical site (for example, the head) is applied to the one untransmitted medical image stored in the CT device 2 so as to detect the specific anatomical site. A detection result may be region information regarding the detected anatomical site or may be textual information. Thereafter, progress information on the transmission process at the current time is generated based on the detection result.

In step S402, the medical information processing apparatus 1 displays progress information by executing the display control function 103 (the process corresponds to step S303). Specifically, the progress information on the transmission process associated with the untransmitted medical image is displayed on the display device 14.

In step S403, the medical information processing apparatus 1 executes the termination function 104 to determine whether or not a communication termination instruction has been received from the user (the process corresponds to step S305). The communication termination instruction is input to the medical information processing apparatus 1 via the input interface 12 by the user at an arbitrary time while the medical information processing apparatus 1 repeatedly executes steps S401 to S402. The input instruction waits for determination until it is processed in step S403. Where the medical information processing apparatus 1 determines that the instruction has been received (YES in step S403), the process proceeds to step S404. Where it is determined that the instruction has not been received (NO in step S403), the process returns to step S401.

In step S404, the medical information processing apparatus 1 terminates the transmission of the medical image by executing the termination function 104, with the reception of the communication termination instruction from the user as a trigger (the process corresponds to step S305). If the communication termination instruction is received during the processes of steps S401 to S402, the medical information processing apparatus 1 may interrupt the processes and terminate the transmission of the medical image at the time when the communication termination instruction is input. Specifically, the medical information processing apparatus 1 executes a transmission termination process for releasing the association the CT device 2 establishes in step S302.

As can be seen from the above, the medical information processing apparatus 1 is incorporated in the sender of medical images, such as the CT device 2, so that the medical information processing apparatus 1 can generate and display progress information on the transmission process of medical images. Further, the medical information processing apparatus 1 can cause the sender to terminate the transmission of medical images by using a user input or a predetermined condition as a trigger.

### Third Embodiment

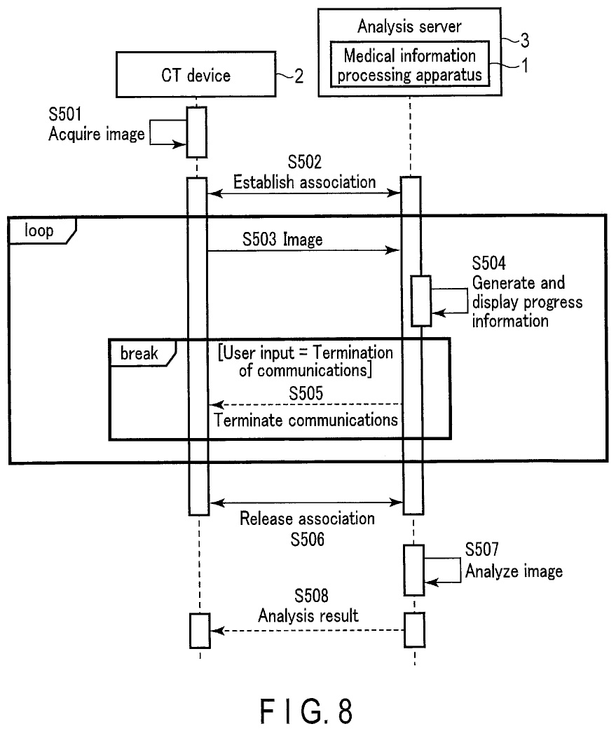

An operation of the medical information processing system according to the third embodiment will be described with reference to FIG. 8. FIG. 8 is a sequence diagram illustrating how the medical information processing system operates according to the third embodiment. In the third embodiment, the medical information processing apparatus 1 is incorporated in the analysis server 3, which is the receiver of medical images. At this time, the medical information processing apparatus 1 displays progress information on the medical image reception process of the analysis server 3, and terminates the reception process of the analysis server 3 in response to a trigger. The analysis server 3 may independently execute all operations of the medical information processing apparatus 1.

In step S501, the CT device 2 acquires a medical image of a subject. Step S501 is similar to step S101.

In step S502, the CT device 2 establishes an association for DICOM communications with reference to the analysis server 3. Step S502 is similar to step S302.

In step S503, the CT device 2 transmits the medical image to the analysis server 3. Step S503 is similar to step S104.

In step S504, the medical information processing apparatus 1 generates and displays progress information on the reception process, based on an anatomical site of the subject included in a received medical image, which is a reception-target medical image the CT device 2 transmits in step S503. For example, the medical information processing apparatus 1 detects the anatomical site of the subject by applying a detection algorithm to one received medical image received by the analysis server 3. Subsequently, based on the detected anatomical site, which is a detection result, progress information on the reception process is generated and displayed. Each time step S504 is repeatedly executed, newly generated progress information may be updated by overwriting already generated progress information.

In step S505, the medical information processing apparatus 1 determines whether or not a communication termination instruction has been received from the user. The communication termination instruction is input to the medical information processing apparatus 1 by the user at an arbitrary time while the medical information processing apparatus 1 repeatedly executes steps S503 to S504. The input instruction waits for determination until it is processed in step S505. Where the medical information processing apparatus 1 determines that the communication termination instruction has been received, the reception of medical images by the analysis server 3 is terminated. Specifically, the medical information processing apparatus 1 transmits to the CT device 2 a transmission termination instruction for releasing the association the CT device 2 establishes in step S502. The medical information processing apparatus 1 may terminate the reception of the medical images at the time when the communication termination instruction is input. On the other hand, where the medical information processing apparatus 1 determines that a communication termination instruction is not received, step S505 is not executed and the process returns to step S503.

In step S506, the CT device 2 releases the association with reference to the analysis server 3, based on the received transmission termination instruction. For example, the CT device 2 requests the analysis server 3 to release the association, and the analysis server 3 makes an association release response. The CT device 2 may release the association without accepting the response of the analysis server 3. Thus, the analysis server 3 can perform image analysis on medical images acquired in units of one association starting from the establishment of the association to the release thereof.

In step S507, the analysis server 3 performs image analysis on the acquired medical images. Step S507 is similar to step S110.

In step S508, the analysis server 3 sends an analysis result to the CT device 2. Step S508 is similar to step S111.

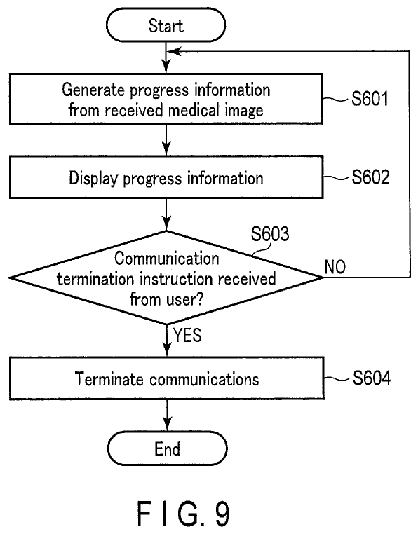

An operation of the medical information processing apparatus according to the third embodiment will be described with reference to FIG. 9. FIG. 9 is a flowchart illustrating how the medical information processing apparatus operates according to the third embodiment.

In step S601, the medical information processing apparatus 1 executes the generation function 102 to generate progress information on the reception process, based on an anatomical site included in a received medical image, which is a reception-target medical image (the process corresponds to step S504). For example, an algorithm for detecting a specific anatomical site (for example, the head) is applied to a medical image received by the analysis server 3 so as to detect the specific anatomical site. A detection result may be region information regarding the detected anatomical site or may be textual information. Thereafter, progress information on the reception process at the current time is generated based on the detection result.

In step S602, the medical information processing apparatus 1 displays progress information by executing the display control function 103 (the process corresponds to step S504). Specifically, the progress information on the reception process associated with the received medical image is displayed on the display device 14.

In step S603, the medical information processing apparatus 1 executes the termination function 104 to determine whether or not a communication termination instruction has been received from the user (the process corresponds to step S505). The communication termination instruction is input to the medical information processing apparatus 1 via the input interface 12 by the user at an arbitrary time while the medical information processing apparatus 1 repeatedly executes steps S601 to S602. The input instruction waits for determination until it is processed in step S603. Where the medical information processing apparatus 1 determines that the instruction has been received (YES in step S603), the process proceeds to step S604. Where it is determined that the instruction has not been received (NO in step S603), the process returns to step S601.

In step S604, the medical information processing apparatus 1 executes the termination function 104 to terminate the reception of the medical image, with the reception of the communication termination instruction from the user as a trigger (the process corresponds to step S505). The medical information processing apparatus 1 may interrupt the process and terminate the reception of the medical image at the time when the communication termination instruction is input. Specifically, the medical information processing apparatus 1 transmits to the CT device 2 a transmission termination instruction for releasing the association the CT device 2 establishes in step S502.

As can be seen from the above, the medical information processing apparatus 1 is incorporated in the receiver of medical images, such as the analysis server 3, so that the medical information processing apparatus 1 can display progress information on the reception process of medical images. Further, the medical information processing apparatus 1 can cause the receiver to terminate the reception of medical images by using a user input or a predetermined condition as a trigger.

As described above, the medical information processing apparatus according to the embodiment generates and displays progress information on a communication process, based on the anatomical site included in a communication-target medical image. The medical information processing apparatus displays the progress information on the communication process of a medical image transmitted from the sender to the receiver, in real time, so that the user can determine to which anatomical site of the subject a communication-target medical image taken at the present time corresponds. Further, since the medical information processing apparatus terminates the communication of medical images where there is a user input or where a predetermined condition is satisfied, the analysis server can immediately start desired image analysis. Thus, the analysis server can start image analysis as soon as the medical images which are included among a large number of medical images related to the subject, such as whole-body CT images, and which are necessary for analysis are available. For example, analysis of urgent brain damage, such as an acute cerebral infarction, can be performed without delay, and improved prognosis of the patient can be expected.

According to at least one of the embodiments described above, the analysis of medical images in a desired unit can be supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information processing apparatus comprising:
processing circuitry configured to:
generate progress information on a communication process, based on an anatomical site of a subject included in a communication-target medical image, in the communication process of the medical image related to the subject;
display the progress information; and
terminate the communication process in response to a trigger that is a signal for terminating the communication process.

2. The medical information processing apparatus according to claim 1, wherein the processing circuitry is further configured to terminate the communication process, with a user input as the trigger.

3. The medical information processing apparatus according to claim 1, wherein the processing circuitry is further configured to terminate the communication process, with a satisfied condition as the trigger.

4. The medical information processing apparatus according to claim 3, wherein the condition is that the communication-target medical image contains a site other than a specific anatomical site of the subject.

5. The medical information processing apparatus according to claim 1, wherein the processing circuitry is further configured to display the progress information on an anatomical atlas.

6. The medical information processing apparatus according to claim 5, wherein the processing circuitry is further configured to display the progress information by means of a bar indicating a position where in the anatomical atlas the communication-target medical image corresponds to.

7. The medical information processing apparatus according to claim 5, wherein the processing circuitry is further configured to display the progress information by coloring a region on the anatomical atlas corresponding to a communicated medical image.

8. The medical information processing apparatus according to claim 1, wherein the communication process is a transmission process related to the medical image, and
the processing circuitry is further configured to generate progress information on the transmission process, based on the anatomical site of the subject included in a transmission-target medical image.

9. The medical information processing apparatus according to claim 1, wherein the communication process is a reception process related to the medical image, and
the processing circuitry is further configured to generate progress information on the reception process, based on the anatomical site of the subject included in a reception-target medical image.

10. A medical information processing method comprising:
generating progress information on a communication process, based on an anatomical site of a subject included in a communication-target medical image, in the communication process of the medical image related to the subject;
displaying the progress information; and
terminating the communication process in response to a trigger that is a signal for terminating the communication process.
